(19) United States
(12) Patent Application Publication
(10) Pub. No.: US 2006/0155373 A1
(43) Pub. Date: Jul. 13, 2006

(54) Introacular lens assembly with distinguishing structure

(76) Inventor: Henry M. Israel, Bnei Brak (IL)

Correspondence Address: DEKEL PATENT LTD., DAVID KLEIN, BEIT HAROF'IM, 18 MENUHA VENAHALA STREET, ROOM 27, REHOVOT, 76209, IL

(21) Appl. No.: 11/033,818

(22) Filed: Jan. 13, 2005

(51) Int. Cl. A61F 2/16

(52) U.S. Cl. 623/6.4; 623/6.49

(57) ABSTRACT

An intraocular lens assembly including an optic connected to an ocular interface element by at least one support member, wherein the at least one support member has a dimension that varies in a direction that distinguishes an anterior side of the optic from a posterior side of the optic, which may be an anterior-posterior-asymmetric optic.

### FIELD OF THE INVENTION

The present invention relates generally to intraocular lens (IOL) assemblies and particularly to an IOL assembly with supporting structure for an optic that distinguishes between the anterior and posterior sides of the optic.

### BACKGROUND OF THE INVENTION

Aspheric intraocular lens (IOL) assemblies have been developed that comprise an optic with aspheric surfaces. As is known in the art, visual acuity of aspheric intraocular lenses may be significantly better than spherical intraocular lenses above certain pupil openings as shown by a modulation transfer function (MTF) analysis, which describes the loss of visual contrast caused by an optical system for various spatial frequencies and pupil diameters.

An aspheric IOL is just one kind of IOL that has an anterior surface which is different from the posterior surface. (In general, throughout the specification and claims, an optic with different anterior and posterior surfaces will be referred to as an anterior-posterior-asymmetric optic.) Another asymmetric optic is a biconvex optic whose anterior side has a different convexity than the posterior side. Another example is a convexoplano optic, with one side being convex and the other planar.

One problem associated with such IOLs is the importance of placing the IOL correctly in the capsular bag, so that the anterior side of the IOL is indeed facing anteriorly and the posterior side is facing posteriorly. The problem is complicated by the small size of the IOL.

### SUMMARY OF THE INVENTION

The present invention seeks to provide an IOL assembly with supporting structure for an optic that distinguishes between the anterior and posterior sides of the optic, as is described more in detail hereinbelow.

There is provided in accordance with an embodiment of the present invention an intraocular lens assembly including an optic connected to an ocular interface element by at least one support member, wherein the at least one support member has a dimension that varies in a direction that distinguishes an anterior side of the optic from a posterior side of the optic, which may be an anterior-posterior-asymmetric optic.

In accordance with an embodiment of the present invention, the support member may include a strut radially extending from the optic to an inner periphery of an at least partial ring, wherein a junction of the strut and the optic is thicker on one side of the strut than the other side of the strut, wherein the thicker side points in a direction that distinguishes the anterior side from the posterior side of the optic.

### BRIEF DESCRIPTION OF DRAWINGS

The present invention will be further understood and appreciated from the following detailed description taken in conjunction with the drawings in which:

FIGS. 1-3 are simplified anterior-view, perspective and sectional illustrations, respectively, of an IOL assembly, constructed and operative in accordance with an embodiment of the invention, wherein FIG. 3 is taken along lines A-A in FIG. 1.

### DETAILED DESCRIPTION OF EMBODIMENTS

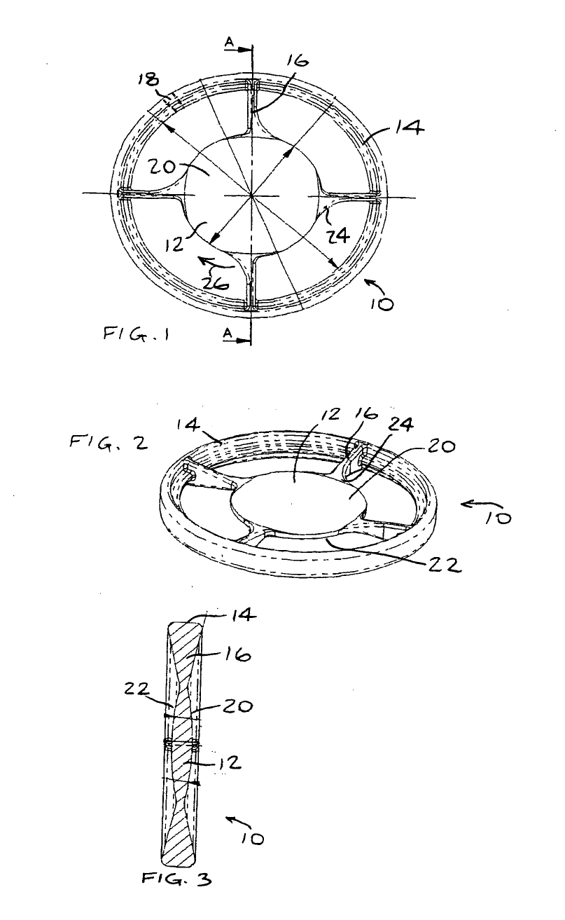

Reference is now made to FIGS. 1-3, which illustrate an intraocular lens (IOL) assembly 10 constructed and operative in accordance with a preferred embodiment of the present invention.

IOL assembly 10 may include an optic 12 connected to an ocular interface element 14 by one or more support members 16. In the illustrated embodiment, the ocular interface element 14 is shaped generally like a ring, which may optionally have one or more splits 18 (this option being shown in FIG. 1 only). In alternative embodiments, ocular interface element 14 may comprise a wire or plate haptic, for example. In the description that follows, ocular interface element 14 will be referred to alternatively as ring 14.

IOL assembly 10 is preferably constructed of a clear, transparent, biologically compatible material, such as but not limited to, polymethylmethacrylate (PMMA), silicone, silicone rubber, collagen, hydrogel, hyaluronic acid, polysulfones, thermolabile materials and other relatively hard or relatively soft and flexible biologically inert optical materials.

Optic 12 may be an anterior-posterior-asymmetric optic, such as but not limited to, aspheric optic, asymmetric biconvex optic or convexoplano optic, for example. One or all of the support members 16 may have a dimension that varies in a direction that distinguishes an anterior side 20 of optic 12 from a posterior side 22 of optic 12, a non-limiting example of which is now described.

In the illustrated embodiment, the support member 16 includes a strut radially extending from the optic 12 to an inner periphery of ring 14. The junction of the strut and the optic 12 is thicker on one side of the strut (as indicated by reference numeral 24) than the other side of the strut. The thicker side 24 points in a direction that distinguishes the anterior side 20 from the posterior side 22 of optic 12. For example, the thicker side 24 may have an arrowhead shape, which points in a clockwise direction (indicated by arrow 26) as viewed from the anterior side 20 of the optic. A surgeon can easily discern the difference between the anterior side 20 and the posterior side 22 of optic 12 by observing the direction in which the thicker side 24 points. Namely, the thicker side 24 points clockwise for the anterior side 20 and counterclockwise for the posterior side 22. In this manner, the surgeon can safely and correctly insert the IOL assembly 10 in an aphakic eye. It is appreciated that support members 16 may be shaped in other manners to provide distinction between the anterior and posterior sides 20 and 22 of the optic 12.

It is appreciated that various features of the invention which are, for clarity, described in the contexts of separate embodiments, may also be provided in combination in a single embodiment. Conversely, various features of the invention which are, for brevity, described in the context of a single embodiment, may also be provided separately or in any suitable subcombination.

1. An intraocular lens assembly comprising:
an optic connected to an ocular interface element by at least one support member, wherein said at least one support member has a dimension that varies in a direction that distinguishes an anterior side of said optic from a posterior side of said optic.

2. The intraocular lens assembly according to claim 1, wherein said optic comprises an anterior-posterior-asymmetric optic.

3. The intraocular lens assembly according to claim 1, wherein said ocular interface element comprises an at least partial ring.

4. The intraocular lens assembly according to claim 3, wherein said at least one support member comprises a strut radially extending from said optic to an inner periphery of said at least partial ring, wherein a junction of said strut and said optic is thicker on one side of the strut than the other side of the strut, wherein the thicker side points in a direction that distinguished the anterior side from the posterior side of said optic.

5. The intraocular lens assembly according to claim 4, wherein the thicker side of said strut has an arrowhead shape.

6. The intraocular lens assembly according to claim 4, wherein the thicker side of said strut points in a clockwise direction as viewed from the anterior side of said optic.
